SEQUENCE LISTING (43 sequences)

SEQ ID NO: 1 - protein, 15 aa, Artificial Sequence
  Synthetic sequence
LVLEVAQHLGESTVR

SEQ ID NO: 2 - protein, 16 aa, Artificial Sequence
  Synthetic sequence
IMDPNIVGSEHYDVAR

SEQ ID NO: 3 - protein, 14 aa, Artificial Sequence
  Synthetic sequence
AHGGYSVFAGVGER

SEQ ID NO: 4 - protein, 18 aa, Artificial Sequence
  Synthetic sequence
EGNDLYHEMIESGVINLK

SEQ ID NO: 5 - protein, 11 aa, Artificial Sequence
  Synthetic sequence
HALIIYDDLSK

SEQ ID NO: 6 - protein, 12 aa, Artificial Sequence
  Synthetic sequence
HVIQSISAQQEK

SEQ ID NO: 7 - protein, 8 aa, Artificial Sequence
  Synthetic sequence
HYLFDVQR

SEQ ID NO: 8 - protein, 12 aa, Artificial Sequence
  Synthetic sequence
LDYHISVQDMMR

SEQ ID NO: 9 - protein, 12 aa, Artificial Sequence
  Synthetic sequence
NCAQFVTGSQAR

SEQ ID NO: 10 - protein, 9 aa, Artificial Sequence
  Synthetic sequence
SWNETFHTR

SEQ ID NO: 11 - protein, 11 aa, Artificial Sequence
  Synthetic sequence
THSDQFLVSFK

SEQ ID NO: 12 - protein, 10 aa, Artificial Sequence
  Synthetic sequence
HLIIGVSSDR

SEQ ID NO: 13 - protein, 22 aa, Artificial Sequence
  Synthetic sequence
GEVPCTVTTAFPLDEAVLSELK

SEQ ID NO: 14 - protein, 10 aa, Artificial Sequence
  Synthetic sequence
LLLQVQHASK

SEQ ID NO: 15 - protein, 14 aa, Artificial Sequence
  Synthetic sequence
GADIMYTGTVDCWR

SEQ ID NO: 16 - protein, 10 aa, Artificial Sequence
  Synthetic sequence
IFSSEHDIFR

SEQ ID NO: 17 - protein, 20 aa, Artificial Sequence
  Synthetic sequence
CIGAIAMTEPGAGSDLQGVR

SEQ ID NO: 18 - protein, 15 aa, Artificial Sequence
  Synthetic sequence
LEDTLWAGLTDQHVK

SEQ ID NO: 19 - protein, 16 aa, Artificial Sequence
  Synthetic sequence
IVYGHLDDPANQEIER

SEQ ID NO: 20 - protein, 11 aa, Artificial Sequence
  Synthetic sequence
DYPLPDVAHVK

SEQ ID NO: 21 - protein, 8 aa, Artificial Sequence
  Synthetic sequence
LVPYQMVH

SEQ ID NO: 22 - protein, 13 aa, Artificial Sequence
  Synthetic sequence
LFQEDNGMPVHLK

SEQ ID NO: 23 - protein, 9 aa, Artificial Sequence
  Synthetic sequence
SHYEEGPGK

SEQ ID NO: 24 - protein, 13 aa, Artificial Sequence
  Synthetic sequence
NALANPLYCPDYR

SEQ ID NO: 25 - protein, 7 aa, Artificial Sequence
  Synthetic sequence
HNGYDPR

SEQ ID NO: 26 - protein, 13 aa, Artificial Sequence
  Synthetic sequence
GCDVVVIPAGVPR

SEQ ID NO: 27 - protein, 15 aa, Artificial Sequence
  Synthetic sequence
ETECTYFSTPLLLGK

SEQ ID NO: 28 - protein, 12 aa, Artificial Sequence
  Synthetic sequence
VNVPVIGGHAGK

SEQ ID NO: 29 - protein, 8 aa, Artificial Sequence
  Synthetic sequence
HGVYNPNK

SEQ ID NO: 30 - protein, 29 aa, Artificial Sequence
  Synthetic sequence
VSDAISTQYPVVDHEFDAVVVGAGGAGLR

SEQ ID NO: 31 - protein, 15 aa, Artificial Sequence
  Synthetic sequence
IIHLTDDSFDTDVLK

SEQ ID NO: 32 - protein, 16 aa, Artificial Sequence
  Synthetic sequence
MIAPILDEIADEYQGK

SEQ ID NO: 33 - protein, 12 aa, Artificial Sequence
  Synthetic sequence
LNIDQNPGTAPK

SEQ ID NO: 34 - protein, 9 aa, Artificial Sequence
  Synthetic sequence
GIPTLLLFK

SEQ ID NO: 35 - protein, 18 aa, Artificial Sequence
  Synthetic sequence
SDKIIHLTDDSFDTDVLK

SEQ ID NO: 36 - protein, 17 aa, Artificial Sequence
  Synthetic sequence
VGLIGSCTNSSYEDMGR

SEQ ID NO: 37 - protein, 8 aa, Artificial Sequence
  Synthetic sequence
EHAALEPR

SEQ ID NO: 38 - protein, 10 aa, Artificial Sequence
  Synthetic sequence
HAALLGGGQR

SEQ ID NO: 39 - protein, 7 aa, Artificial Sequence
  Synthetic sequence
NYPEGHR

SEQ ID NO: 40 - protein, 9 aa, Artificial Sequence
  Synthetic sequence
KHNFTPLAR

SEQ ID NO: 41 - protein, 12 aa, Artificial Sequence
  Synthetic sequence
MGHAGAIIAGGK

SEQ ID NO: 42 - protein, 8 aa, Artificial Sequence
  Synthetic sequence
THINYGVK

SEQ ID NO: 43 - protein, 14 aa, Artificial Sequence
  Synthetic sequence
GVIALCIEDGSIHR

(19) United States
(12) Patent Application Publication
(10) Pub. No.: US 2009/0004684 A1
(43) Pub. Date: Jan. 1, 2009

(54) Compositions and Methods for Detection and Quantification of Protein Oxidation

(76) Inventors: Claudia Susanne Maier, Corvallis, OR (US); Jan Frederik Stevens, Corvallis, OR (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/125,584

(22) Filed: May 22, 2008

Related U.S. Application Data
(60) Provisional application No. 60/931,555, filed on May 23, 2007.

(51) Int. Cl. C12Q 1/37; C07K 1/107; G01N 31/22; G01N 33/48

(52) U.S. Cl. 435/23; 530/409; 436/8; 436/86

(57) ABSTRACT

Compositions and methods for detecting carbonyl functional groups on polypeptides are disclosed.

This application claims priority under 35 U.S.C. §119(e) to U.S. Provisional Patent Application No. 60/931,555, filed on May 23, 2007. The foregoing application is incorporated by reference herein.

Pursuant to 35 U.S.C. Section 202(c), it is acknowledged that the United States Government has certain rights in the invention described herein, which was made in part with funds from the National Institutes of Health Grant No. R01 AG025372.

### FIELD OF THE INVENTION

The present invention relates generally to the detection of protein oxidation. Specifically, compositions and methods are provided for detecting and quantifying oxidation products.

### BACKGROUND OF THE INVENTION

Several publications and patent documents are cited throughout the specification in order to describe the state of the art to which this invention pertains. Each of these citations is incorporated herein by reference as though set forth in full.

About 5 million Americans are suffering from congestive heart failure, with 550,000 new cases diagnosed each year. As the population of elderly grows over the next decades and the health-care burden sky-rockets there is an increasing pressure in understanding the mechanisms leading to age-related cardiac dysfunction. Myocardial stiffness and atrophy are conditions of mitochondrial-related mechanisms of dysfunction in the aging heart. Mitochondrial oxidative stress contributes to cardiac atrophy via necrosis or apoptosis. A better understanding of the oxidative stress-mediated changes to the proteome that occur in the mitochondrion as a consequence of aging will translate to treatment strategies to maintain, hold or restore myocardial function in the elderly.

Mitochondria are the vital organelles responsible for providing cellular energy through oxidative phosphorylation. It has been estimated that under normal conditions, up to 2% of oxygen escapes from the respiratory chain in the form of superoxide, a reactive oxygen species (ROS) that can initiate radical-mediated oxygenations (Boveris et al. (1972) Biochem. J., 128:617-630). ROS can chemically modify a variety of biological molecules, including polyunsaturated acyl chains of membrane phospholipids to generate a group of lipid hydroperoxides (Schneider et al. (2001) J. Biol. Chem., 276:20831-20838). Subsequent reactions of these lipid hydroperoxides give rise to a variety of α,β-unsaturated aldehydic products, such as keto aldehydes, 2-alkenals, and 4-hydroxy-2-alkenals (Uchida, K. (2003) Prog. Lipid Res., 42:318-343; Uchida, K. (1999) Trends Cardiovasc. Med., 9:109-113). Especially, 4-hydroxy-2-alkenals, such as 4-hydroxy-2-nonenal (HNE) and 4-hydroxy-2-hexenal (HHE), represent prominent aldehydes generated during lipid peroxidation, which are highly reactive toward nucleophilic amino acid residues and contribute to cellular damage associated with oxidative stress (Segall et al. (1985) Science 229:472-475; Yamada et al. (2004) Lipid Res., 45:626-634; Grasse et al. (1985) Toxicol. Lett., 29:43-49; Shibata et al. (2004) Brain Res., 1019:170-177; Lee et al. (2004) Eur. J. Biochem., 271:1339-1347; Zarkovic, K. (2003) Mol. Aspects Med., 24:293-303; Grune et al. (2003) J. Mol. Aspects Med., 24:195-204). Modifications by 2-alkenals were found to occur mainly on cysteine, histidine, and lysine residues preferentially via Michael-type addition reactions preserving the aldehyde functionality on the modified protein, rather than by Schiff's base formation (Esterbauer et al. (1991) Free Radical Biol. Med., 11:81-128; Carini et al. (2004) Mass Spectrom. Rev., 23:281-305; Schaur, R. J. (2003) Mol. Aspects Med., 24:149-159). Carbonyl groups are also introduced into peptides/polypeptides/proteins by metal-catalyzed oxidation of amino acid side chains and oxidation of the polypeptide backbone accompanied by peptide bond cleavage (Stadtman et al. (1997) Chem. Res. Toxicol., 10:485-494).

### SUMMARY OF THE INVENTION

In accordance with the present invention, compounds for the detection of carbonyl group(s) in a polypeptide are provided. The compound of the instant invention may comprise a) a carbonyl reactive moiety which forms a bond with the polypeptide via an aldehyde or ketone group; b) an affinity tag; and c) a linker region which operably links the carbonyl reactive moiety to the affinity tag. In a particular embodiment, the carbonyl reactive moiety is a hydrazide or a hydroxylamine. In still another embodiment, the compound comprises an isotope (e.g., a stable isotope), particularly in the linker region.

In accordance with another aspect of the instant invention, compositions comprising at least one compound of the invention and a carrier are provided. In still another aspect, the composition is contained within a kit.

According to another aspect of the instant invention, methods for detecting and/or quantitating at least one ketone or aldehyde group on a polypeptide or detecting and/or quantitating polypeptides which comprise at least one ketone or aldehyde in a composition are provided. In a particular embodiment, the methods comprise a) obtaining a composition comprising at least one polypeptide, b) fragmenting the polypeptides in the sample into peptides, c) contacting the peptides with at least one compound of the instant invention, and d) isolating peptides linked to the compound by affinity chromatography. In another embodiment, the methods comprise contacting the polypeptides with at least one compound of the instant invention prior to fragmentation. The presence of peptides linked to the compound of the instant invention indicates the presence at least one ketone or aldehyde group on the polypeptide which comprises the modified peptide. In another embodiment, the methods further comprise determining the sequence of the peptide linked to the compound. In still another embodiment, the labeling step is performed wherein one portion of the composition comprising the polypeptides or fragments after cleavage is reacted with a compound of the instant invention which does not comprise a stable isotope and contacting the other (e.g., equivalent) portion of the composition with a compound of the instant invention (e.g., the same compound) which comprises a stable isotope, thereby allowing for quantitating the amount of polypeptide(s) comprising a carbonyl group by performing isotope dilution mass spectrometry.

### BRIEF DESCRIPTIONS OF THE DRAWING

FIG. 1A provides a schematic of an isotope-coded affinity tag (ICAT) associated reaction and a hydrazide-functionalized isotope-coded affinity tag (HICAT) associated reaction. FIG. 1B provides a scheme for the synthesis and structure of a hydrazide-functionalized isotope-coded affinity tag (HICAT).

FIG. 2 provides a scheme for using the isotopomeric probes ¹²C₄- and ¹³C₄-HICAT for the identification, characterization, and quantification of oxylipid-protein conjugates.

FIGS. 3A and 3B provide MALDI-MS spectra of E. coli thioredoxin (Trx) after 4-hydroxy-2-nonenal (HNE) modification (FIG. 3A) and HICAT labeling (FIG. 3B). In FIG. 3A, the difference of ˜158 Da between the mass peak at m/z 11 671.5 and 11 828.9 indicates Michael-type addition of HNE. A small fraction of the protein was found to be modified by two molecules of HNE (TRX-(HNE)₂, MH⁺ 11 985 Da). In FIG. 3B, the difference of ˜630 Da between the mass peak at m/z 12 303.9 and 11 674.3 provides evidence of the successful labeling of the HNE moiety by HICAT. The peaks at m/z 12 457 and 12 934 indicate labeling of TRX-(HNE)₂ by one and two HICAT molecules, respectively.

FIGS. 4A and 4B demonstrate the affinity enrichment of the HICAT-labeled HNE-peptide conjugate. FIG. 4A provides a MALDI mass spectrum of the unfractionated tryptic digest of HNE-modified thioredoxin. The tryptic HICAT-labeled peptide T2-HNE-HICAT is visible at m/z 2360.2. The following tryptic peptides containing no HICAT were also observed: T2 (1731.9 Da) and T2-HNE (1888.05 Da), IIHLTDDSFDTDVLK (SEQ ID NO: 31); T4 (1805.9 Da) and T4OX (1821.9 Da), MIAPILDEIADEYQGK (SEQ ID NO: 32); T6 (1267.7 Da), LNIDQNPGTAPK (SEQ ID NO: 33); and T8 (1001.7 Da), GIPTLLLFK (SEQ ID NO: 34). The peak at m/z 2220.2, annotated with #, represents the miscleaved N-terminal peptide T1,2 (aa 1-18) modified by reduced HNE (C₉H₁₈O₂, 158 Da). The tandem mass spectrum that yielded the identification of this peptide is shown in FIG. 4C. FIG. 4B provides a MALDI mass spectrum of the enriched fraction containing the HICAT-labeled HNE conjugate of peptide T2. The other minor peaks did not yield peptide identifications using tandem mass spectral data in combination with Mascot searches. FIG. 4C provides a MALDI tandem mass spectrum of the tentatively assigned peptide T1,2 (aa 1-18) (SEQ ID NO: 35) modified by reduced HNE (C₉H₁₈O₂, 158 Da). The observation of fragment ion y₁₅ and the b-ion series, b₆-b₁₇, indicates that the lipid modification is located near the N-terminus of the peptide. Although the N-terminal amino group has the more favorable pKa value, the observed mis-cleavage on Lys-3 indicates that the ε-amino group of Lys-3 was modified by HNE. Reduction with NaCNBH3 caused the reduction of the HNE moiety to the respective alcohol, termed reduced HNE (C₉H₁₈O₂, 158 Da). Fragment ions marked with an asterisk * retained the lipid moiety during high-energy collision induced fragmentation. The fragment ion annotated with # is tentatively assigned as an internal fragment ion. This spectrum obtained a Mascot score of 84. Notably, Mascot scores higher than 30 are usually considered as sufficient for positive identification.

FIG. 5A provides a schematic of a HICAT-labeled HNE-modified peptide T2 from E. coli Trx and SEQ ID NO: 31. FIG. 5B provides a MALDI tandem mass spectrometric identification of the HICAT-labeled HNE-modified peptide T2 from E. coli Trx. Fragment ions marked with an asterisk (*) retained the HICAT-HNE moiety during high-energy collision-induced fragmentation. The ion at m/z 738.5 corresponds to the immonium ion of the HICAT-labeled HNE-conjugated histidine residue. The prominent ion at m/z 1731.8 indicates neutral loss of the HICAT-HNE moiety. This spectrum obtained a Mascot score of 50.

FIGS. 6A and 6B provide MALDI-MS survey scans of the tryptic digest of ¹²C₄/¹³C₄-HICAT-tagged HNE-conjugated TRX after affinity chromatography (FIG. 6A) and MALDI-MS/MS analysis of the ¹²C₄/¹³C₄-HICAT-labeled tryptic peptide T2 (FIG. 6B). The inset of FIG. 6B shows the isotopomeric pair of the b₇ fragment ion. SEQ ID NO: 31 is also provided in FIG. 6B. Fragment ions marked with an asterisk (*) retained the HICAT-HNE moiety during high-energy collision-induced fragmentation.

FIG. 7 provides a MALDI tandem mass spectrometric identification of the HICAT-labeled in vitro HNE-modified peptide (aa 92-104) (SEQ ID NO: 26) from malate dehydrogenase (MDHM_RAT).

FIGS. 8A and 8B provide MALDI (FIG. 8A) and ESI (FIG. 8B) tandem mass spectrometric identification of the ¹²C-HICAT-labeled in vitro HNE-modified peptide (aa 95-109) from ATP synthase β-chain (ATPB_RAT). SEQ ID NO: 1 is provided in FIGS. 8A and 8B.

FIGS. 9A and 9B demonstrate the reproducibility of relative peak area measurements illustrated for the ¹²C₄- and ¹³C₄-HICAT-labeled HNE-modified peptide (amino acids (aa) 226-239) of ATPβ_RAT. FIG. 9A provides reconstructed ion chromatograms using Peak Explorer software version 3.0 for a ¹²C- and ¹³C-tagged peptide pair that was identified as peptide aa 226-239 of ATPβ_RAT. Each fraction number corresponds to 20 seconds of eluting time, in which the ¹²C₄- and ¹³C₄-HICAT HNE-modified peptides co-eluted. FIG. 9B provides MALDI survey mass spectra for three samples prepared from rat heart mitochondria. The m/z region of the ¹²C/¹³C peptide pair is shown.

FIG. 10 provides MALDI-MS analysis of an HICAT-labeled in vivo acrolein-peptide conjugate (SEQ ID NO: 15) from ADP/ATP translocase 1 (ADT1_RAT, aa 245-258) found in rat heart mitochondria. FIG. 10A provides a tandem mass spectrum of the ¹²C₄-HICAT-labeled peptide ion with m/z 2116.0. FIG. 10B provides MS survey scans of three independently performed HICAT labeling experiments showing the ion traces of the ¹²C₄-HICAT-labeled peptide ion at m/z 2116.0 (from young animals) and the ¹³C₄-HICAT-labeled peptide ion at m/z 2120.0 (from old animals). An average isotope cluster area ratio of 1.45±0.19 was determined.

FIG. 11 provides MALDI tandem mass spectra of the HICAT-labeled aldehyde-modified peptide UQCR2_RAT, aa 183-185. Cys-191 was identified as target site of modification by 4-hydroxyhexenal (HHE) (top), acrolein (middle), and 4-oxononenal (bottom). P* annotate the respective precursor ions that were used for tandem mass spectrometry. Peptide fragmentation is annotated as b- and y-ions. Fragment ions annotated with F1, F2, and F3 originate from the HICAT tag and were used as additional “indicator” ions aiding in the identification of HICAT-labeled peptides in complex mitochondrial peptide mixtures.

### DETAILED DESCRIPTION OF THE INVENTION

Herein, a new analytical method is described for identifying, characterizing, and quantifying carbonyl-containing peptides (e.g., oxylipid-conjugated proteins) and carbonyl-containing peptides generated by, for example, amino acid side-chain oxidation and oxidative peptide bond cleavages, in a mixture (e.g., a complex peptide mixture) that makes use of a new probe: HICAT, a hydrazide-functionalized isotope-coded affinity tag, for the selective isolation and analysis of proteins modified by α,β-unsaturated aldehydes and ketones. Protein oxidation (also referred as protein carbonylation) is chemically diverse. Some examples of oxidative modification reactions leading to the introduction of carbonyl groups into proteins/peptides are a) formation of protein/peptide adducts with reactive lipid peroxidation products, b) oxidative modifications of amino acid side-chains (e.g., Lys, Arg, Pro) and c) oxidative peptide bond cleavages.

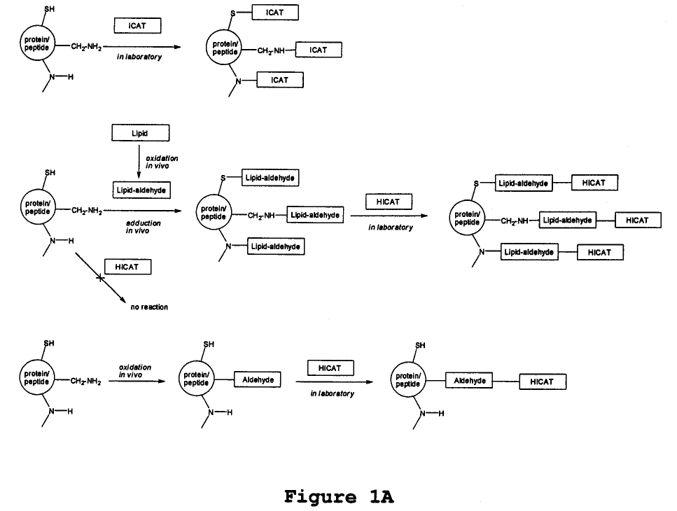

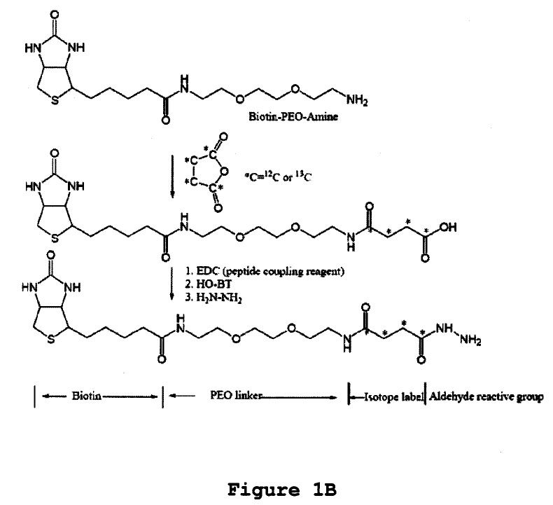

The HICAT reagents consist of three functional components (FIG. 1). The first is a reactive moiety (e.g., a hydrazide or hydroxylamine) that will react with a carbonyl functional group, such as a ketone or aldehyde group, to form, e.g., a hydrazone. The aldehydes and ketones may arise from the adduction of lipid-derived 2-alkenals with proteins. The second component is a linker, such as a succinic anhydride-derived linker, that is present in a ¹²C₄ (isotopically normal) or ¹³C₄ (isotopically heavy) form and provides the basis of relative quantification of protein-alkenal adducts and other carbonyl-modified peptides. The isotope-coded linker allows relative quantification of protein-alkenal adducts by using isotope dilution mass spectrometry. An affinity tag (e.g., a biotin moiety), the third component, enables the enrichment of HICAT-labeled peptides via affinity chromatography (e.g., avidin chromatography).

An isotopically coded affinity probe was developed and evaluated for the characterization and quantification of proteins adducted by 2-alkenals derived from lipid peroxidation (LPO) processes and other carbonyl-modified proteins generated by oxidation of amino acid side-chains and oxidative peptide bond cleavage. Lipid-derived 2-alkenals, such as acrolein and 4-hydroxy-2-nonenal (HNE), have the ability to react with cysteine, histidine, and lysine residues in proteins, thus causing protein oxidation and loss of protein function (damage). Oxidative modifications of proteins are difficult to characterize in biological samples by mass spectrometry due to the complexity of protein extracts and the low abundance of adducted proteins. The novel aldehyde-reactive HICAT described herein was found effective for the selective isolation, detection, and quantification of Michael-type adducts of 2-alkenals with proteins using a combination of affinity isolation, nanoLC, and matrix-assisted laser desorption ionization tandem mass spectrometry (MALDI-MS/MS). The chemical and mass spectrometric properties of the new probe are demonstrated on a model protein treated with HNE. The efficacy of HICAT for the analysis of complex samples was tested using preparations of mitochondrial proteins that were modified in vitro with HNE. The potential of the HICAT strategy for the identification, characterization, and quantification of in vivo carbonyl-modified proteins is demonstrated on cardiac mitochondrial protein preparations, in which, for example, the ADP/ATP translocase 1 was found adducted to the 2-alkenals, acrolein and 4-hydroxy-2-hexenal, at Cys-256.

### I. DEFINITIONS

The following definitions are provided to facilitate an understanding of the present invention:

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

The terms “polypeptide” and “protein” are sometimes used interchangeably herein and indicate a molecular chain of amino acids. The term polypeptide encompasses peptides, oligopeptides, and proteins. The terms also include post-expression modifications of the polypeptide, for example, glycosylations, acetylations, phosphorylations and the like. In addition, protein fragments, analogs, mutated or variant proteins, fusion proteins and the like are included within the meaning of polypeptide. The term “peptide” may be used herein to refer to polypeptides of no more than about 50 amino acids.

As used herein, “diagnosis” refers to providing any type of diagnostic information, including, but not limited to, whether a subject is likely to have a condition, information related to the nature or classification of the condition, information related to prognosis and/or information useful in selecting an appropriate treatment. As used herein, “diagnostic information” or information for use in diagnosis is any information that is useful in determining whether a patient has a disease or condition and/or in classifying the disease or condition into a phenotypic category or any category having significance with regards to the prognosis of or likely response to treatment (either treatment in general or any particular treatment) of the disease or condition.

Generally, “cardiovascular” diseases or disorders refer to the class of diseases or disorders that involve the heart and/or blood vessels.

“Pharmaceutically acceptable” indicates approval by a regulatory agency of the Federal or a state government or listed in the U.S. Pharmacopeia or other generally recognized pharmacopeia for use in animals, and more particularly in humans. Pharmaceutically acceptable carriers are described in “Remington's Pharmaceutical Sciences” by E. W. Martin (Mack Publishing Co., Easton, Pa.); Gennaro, A. R., Remington: The Science and Practice of Pharmacy, 20th Edition, (Lippincott, Williams and Wilkins), 2000; Liberman, et al., Eds., Pharmaceutical Dosage Forms, Marcel Decker, New York, N.Y., 1980; and Kibbe, et al., Eds., Handbook of Pharmaceutical Excipients (3.sup.rd Ed.), American Pharmaceutical Association, Washington, 1999.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), water, bulking substance (e.g., lactose, mannitol), excipient, auxilliary agent or vehicle with which an active agent of the present invention can be contained.

The term “alkyl,” as employed herein, includes straight, branched, and cyclic chain hydrocarbons containing 1 to about 20 carbons in the normal chain. The hydrocarbon chain of the alkyl groups may be interrupted with one or more oxygen, nitrogen, or sulfur. Examples of suitable alkyl groups include methyl, ethyl, propyl, isopropyl, butyl, t butyl, isobutyl, pentyl, hexyl, isohexyl, heptyl, 4,4 dimethylpentyl, octyl, 2,2,4 trimethylpentyl, nonyl, decyl, the various branched chain isomers thereof, and the like. Each alkyl group may, optionally, be substituted, preferably with 1 to 4 substituents. The alkyls of the instant invent may be “lower alkyls,” i.e. an alkyl which contains 1 to 4 carbons in the hydrocarbon chain. Alkyl substituents include, without limitation, alkyl, alkenyl, halo (such as F, Cl, Br, I), haloalkyl (e.g., CCl₃ or CF₃), alkoxyl, alkylthio, hydroxy, methoxy, carboxyl, oxo, epoxy, alkyloxycarbonyl, alkylcarbonyloxy, amino, carbamoyl (e.g., NH₂C(═O)— or NHRC(═O)—, wherein R is an alkyl), urea (—NHCONH₂), alkylurea, aryl, ether, ester, thioester, nitrile, nitro, amide, carbonyl, carboxylate and thiol. Exemplary cycloalkyls include, without limitation, indanyl and adamantyl. The term “alkenyl” refers to an alkyl comprising one or more carbon to carbon double bonds. In a particular embodiment, the alkyl groups of the instant invention are straight, unsubstituted, hydrocarbon chains.

The term “aryl,” as employed herein, refers to monocyclic and bicyclic aromatic groups containing 6 to 10 carbons in the ring portion. Examples of aryl groups include, without limitation, phenyl, naphthyl, such as 1-naphthyl and 2-naphthyl, indolyl, and pyridyl, such as 3-pyridyl and 4-pyridyl. Aryl groups may be optionally substituted through available carbon atoms, preferably with 1 to about 4 groups. Exemplary substituents may include, but are not limited to, alkyl, halo, haloalkyl, alkoxyl, alkylthio, amino, hydroxyl, methoxy, carboxyl, carboxylate, oxo, ether, ester, epoxy, alkyloxycarbonyl, alkylcarbonyloxy, amino, carbamoyl, urea, alkylurea, thioester, amide, nitro, carbonyl, and thiol. The aromatic groups may be a heteroaryl. “Heteroaryl” refers to an optionally substituted aromatic ring system that includes at least one, and preferably from 1 to about 4 sulfur, oxygen, or nitrogen heteroatom ring members.

### II. HICAT

As stated hereinabove, the hydrazide-functionalized isotope-coded affinity tag (HICAT) of the instant invention comprises three functional components. These components are described hereinbelow.

The first component is a moiety which reacts with a carbonyl functional group, i.e., a group comprising a carbon atom double-bonded to an oxygen atom. In a preferred embodiment, the carbonyl functional group is a ketone or aldehyde group. In a preferred embodiment, the carbonyl functional group-reactive moiety reacts with ketone and/or aldehyde groups to the exclusion of other carbonyl groups such as amides. Carbonyl functional group-reactive moieties include, without limitation, amines, aromatic amines (e.g., 2-aminopyridine, 8-aminonaphthalene-1,3,6-disulfonic acid, 1-aminopyrene-3,6,8-trisulfonic acid, and aminoacridone (e.g., 2-aminoacridone)), hydrazides (—NR—NH₂, wherein R is H or an alkyl, preferably H; the term “hydrazide” includes, without limitation carbonyl hydrazides (—(C═O)NRNH₂), sulfonylhydrazides (—(SO₂)NRNH₂), thiocarbonylhydrazides (—(C═S)NR—NH₂), semicarbazides (—NR(C═O)NRNH₂, wherein the R groups are independent), thiosemicarbazides (—NR(C═S)NRNH₂, wherein the R groups are independent), carbazides (—NRNR(C═O)NRNH₂, wherein the R groups are independent), and thiocarbazides ((—NRNR(C═S)NRNH₂, wherein the R groups are independent)), and hydroxylamines (—O—NH₂) (see also, Haugland et al., Molecular Probes, Inc., Handbook of Fluorescent Probes and Research Chemicals, 7^{th} Edition; and U.S. Patent Application Publication No. 205/0250152). In a preferred embodiment, the carbonyl-reactive moiety is a hydrazide or a hydroxylamine.

The second component is a linker region which links the first and third components. More specifically, the linker region is a chemical moiety comprising a covalent bond or a chain of atoms that covalently attaches the carbonyl functional group-reactive moiety to the affinity tag. In a particular embodiment, the linker may contain from 0 (i.e., bond) to about 100 atoms, preferably from 1 to about 50 atoms. The linker can be linked to any synthetically feasible position of carbonyl functional group-reactive moiety and the affinity tag. In a preferred embodiment the linker is attached at a position which avoids inhibiting the activity of the carbonyl functional group-reactive moiety and avoids blocking the affinity tag from being recognized. Exemplary linkers may comprise at least one optionally substituted; saturated or unsaturated; linear, branched or cyclic alkyl, alkenyl, or aryl group. The linker may also be a polypeptide (e.g., from about 1 to about 20 amino acids). The linker may be biodegradable under physiological environments or conditions. The linker may also be non-degradable and may be a covalent bond or any other chemical structure which is not cleaved under physiological environments or conditions. In a particular embodiment, the linker is a succinic anhydride derived linker. The linker may comprise at least one isotope (e.g., a stable isotope) for detection and quantification. Isotopes include, without limitation, ²H, ¹³C, ¹⁵N, ¹⁷O, ¹⁸O, ³³S, ³⁴S, and ³⁶S. In a preferred embodiment, the isotope is a carbon isotope (e.g., ¹³C).

The third component is an affinity tag. An affinity tag (also referred to as a purification tag) is a tag that can be used to effect the purification of a compound of interest. Affinity tags are well known in the art (see Sambrook et al., 2001, Molecular Cloning, Cold Spring Harbor Laboratory) and include, but are not limited to: biotin, polyhistidine tags (e.g., 6×His), polyarginine tags, glutathione-S-transferase (GST), maltose binding protein (MBP), S-tag, influenza virus HA tag, thioredoxin, staphylococcal protein A tag, the FLAG™ epitope, dihydrofolate reductase (DHFR), an antibody epitope (e.g., a sequence of amino acids specifically recognized and bound by an antibody), the c-myc epitope, and heme binding peptides. In a preferred embodiment, the affinity tag is biotin.

Accordingly, the HICAT molecules of the instant invention comprise at least one carbonyl functional group-reactive moiety operably linked via at least one linker to at least one affinity tag. In a particular embodiment of the instant invention, at least one HICAT molecule and at least one carrier (e.g., a pharmaceutically acceptable carrier) may be contained within a single composition.

In accordance with another aspect of the instant invention, at least one of the compositions of the instant invention may be contained within a kit. The kit may further comprise one or more of the following components: instruction material, vials, tubes, at least one enzyme for digesting polypeptides (e.g., trypsin), buffers (e.g., wash buffers and reaction buffers such as a buffer for the digestion of the peptides and/or a buffer for labeling the peptides with HICAT), positive control (peptides comprising aldehyde and/or ketone groups), negative control (peptides without aldehyde and/or ketone groups), mass spectrometry reagents (e.g., buffers), and affinity chromatography reagents (e.g., buffers, resins such as resins comprising avidin or analogs thereof).

### III. METHODS OF USE

In accordance with one aspect of the instant invention, methods are provided for the detection of carbonyl functional groups (aldehyde and/or ketone groups) in a sample. In a particular embodiment, the method comprises obtaining a sample comprising polypeptides/proteins (e.g., isolating the polypeptides from a cellular extract), fragmenting (e.g., digesting with an enzyme) the proteins in the sample into fragments (e.g., peptides), contacting (reacting) the fragmented proteins with at least one HICAT, and isolating HICAT labeled peptides by affinity chromatography. In a particular embodiment, the isolated HICAT labeled peptides may be further characterized. For example, the sequence of the HICAT labeled peptide may be determined (e.g., by mass spectrometry and other non-mass spectrometry based sequencing techniques). The sequence may subsequently be searched against a sequence database to identify the protein. The relative quantity of the HICAT labeled peptides may also be calculated by determining the ratio of differentially isotope-labeled HICAT by isotope dilution mass spectrometry, optionally preceded by HPLC.

In a particular embodiment, the method of the instant invention comprises obtaining a sample comprising polypeptides (e.g., proteins), reacting the polypeptides with at least one HICAT, fragmenting (e.g., digesting with an enzyme) the polypeptides in the sample into fragments (e.g., peptides), and isolating the HICAT labeled peptides by affinity chromatography.

The sample of the methods of the instant invention may be obtained from a patient. In a particular embodiment, the sample is a protein extract of a biological sample. In yet another embodiment, the sample is myocardial.

The polypeptides of the sample may be digested by at least one protease or digested by other means (e.g., chemical digestion). Proteases include, without limitation, trypsin, serine protease, cysteine protease, aspartic protease, threonine protease and metalloprotease, cathepsin, chymase, alpha-tryptase, beta-trypsase I or II, chymotrypsin, collagenase, factor XII, factor XI, factor CII, factor X, thrombin, protein C, u-plasminogen activator (u-PA), t-plasminogen activator (t-PA), plasmin, plasma kallikrein, papain, cruzain, pronase, pepsin, Lys-C protease, Glu-C protease, and thermolysin. In a preferred embodiment, the protease is trypsin. In yet another particular embodiment, the polypeptides of the sample are completely digested or digested such that the average size of the resultant fragments is about 5 to about 50 amino acids in length, more particularly about 7 to about 30 amino acids.

The affinity purification step of the instant methods will vary based on the affinity tag selected. As stated hereinabove in a preferred embodiment, the affinity tag is biotin or analogs thereof. Accordingly, the biotin tagged HICAT labeled peptides may be purified through the use of biotin binding agents, such as resins comprising avidin, streptavidin, or other forms or analogs of avidin.

In accordance with another aspect of the instant invention, methods are provided for the detection of protein oxidation (including protein damage), such as protein oxidation by lipid oxidation products. In a particular embodiment, the method comprises obtaining a sample comprising proteins, fragmenting (e.g., digesting with an enzyme) the proteins in the sample into fragments (e.g., peptides), reacting the fragmented proteins with at least one HICAT, and isolating HICAT labeled peptides by affinity chromatography. The methods may further comprise quantitating the amount of HICAT labeled peptides, which correlates to protein oxidation, by isotope dilution mass spectrometry. The methods may also further comprise the comparison of an unknown sample with known samples (e.g., a positive control wherein the proteins have been oxidized and/or a negative control wherein the proteins are not oxidized by oxidative stress (e.g., native)) to assess the amount of protein oxidation.

In yet another aspect of the instant invention, methods are provided for diagnosing an increased risk for cardiovascular disease, age-related cardiac disorders, and/or decreased myocardial function. In a particular embodiment, the method comprises obtaining a myocardial sample from an animal (including humans), fragmenting (e.g., digesting with an enzyme) the proteins in the myocardial sample into fragments (e.g., peptides), reacting the fragmented proteins with at least one HICAT, and isolating HICAT labeled peptides by affinity chromatography. The methods may further comprise quantitating the amount of HICAT labeled peptides by isotope dilution mass spectrometry. An increase in HICAT labeling is indicative of protein oxidation (e.g., damage) and, therefore, decreased myocardial function and the presence of age-related cardiac disorders and/or cardiovascular disease. The methods may also further comprise the comparison of an unknown sample with known samples (e.g., a positive control wherein the proteins have been oxidized (e.g., a sample from an old/aged heart) and/or a negative control wherein the proteins are not oxidized by oxidative stress (e.g., a sample from a young heart)) to assess the amount of protein oxidation.

The following example provides illustrative methods of practicing the instant invention, and is not intended to limit the scope of the invention in any way.

### EXAMPLE

### Materials and Methods

### Materials

Thioredoxin and sequencing-grade modified trypsin were purchased from Promega Corporation, Madison, Wis. 4-Hydroxy-2-nonenal (HNE, 10 mg/mL in ethanol) was purchased from Cayman Chemicals Inc., Ann Arbor, Mich. Biotin-PEO-amine (PEO, polyethylene oxide) and coomassie plus protein assay reagent were from Pierce Biotechnology (Rockford, Ill.). Succinic anhydride, ¹³C₄-succinic acid, EDC (1-ethyl-3-[3-dimethylaminopropyl]-carbodiimide), HOBT (1-hydroxybenzotriazole), hydrazine, acetic anhydride, and sodium cyanoborohydride were purchased from Sigma-Aldrich (St. Louis, Mo.). The avidin cartridge system was obtained from Applied Biosystems (Foster City, Calif.). R-Cyano-4-hydroxycinnamic acid was from Sigma Chemicals (St. Louis, Mo.).

### Synthesis of ¹²C₄-HICAT and ¹³C₄-HICAT

The synthesis of HICAT involves three reaction steps starting from the commercially available biotin-PEO-amine (N-[13-amino-4,7,10-trioxadidecanyl]biotin-amide). Succinylation of biotin-PEO-amine was carried out in phosphate buffer at pH 8.5 using a 2-fold molar excess of succinic acid anhydride at room temperature for 1 hour. The product was recovered from the aqueous solution by semi-preparative HPLC (Econosil 10 μm ODS (250 mm×10 mm), from 5% to 100% CH₃CN in 30 minutes, flow rate 4 mL/minute, detection at 215 nm) and subsequent lyophilization with 90% yield. For coupling of the biotin-PEO-succinic acid product to hydrazine (Zhang et al. (2002) J. Org. Chem., 67:9471-9474.), a 2-fold molar excess of EDC and a 1.5-fold molar excess of HOBT (1-hydroxybenzotriazole) were reacted with biotin-PEO-succinic acid at room temperature for 40 minutes, and then, a 10-fold molar excess of hydrazine was added. Reaction proceeded for 30 minutes, and the product, HICAT, was isolated from the reaction mixture by semipreparative HPLC (Vydac C4 (250 mm×10 mm), 6% CH₃CN, flow rate 4 mL/minute, detection at 215 nm) and subsequent lyophilization with 70% yield. Accordingly, the isotopomeric probe ¹³C₄-HICAT was synthesized by using carbon-13 (¹³C)-labeled succinic anhydride. ¹³C₄-succinic anhydride was prepared from ¹³C₄-succinic acid by refluxing in acetic anhydride with over 80% yield according to a procedure described in Furniss et al. (Vogel's Textbook of Practical Organic Chemistry, 5th ed.; 1989; p 695.). The overall yield of the HICAT synthesis was approximately 60%.

### Characterization of ¹²C₄-HICAT and ¹³C₄-HICAT

High-resolution mass spectra of ¹²C₄-HICAT and ¹³C₄-HICAT were acquired with an LCT Classic mass spectrometer equipped with an electrospray ionization (ESI) source (Micromass/Waters, Manchester, U.K.). Mass spectral measurements were taken with the following settings and conditions: capillary voltage 3 kV, sample cone voltage 7 V, extraction cone voltage 5 V, source temperature 80° C., desolvation temperature 120° C., desolvation gas 450 L/hour. The mass spectra of ¹²C₄-HICAT and ¹³C₄-HICAT showed molecular ions, [M+H]⁺, at m/z 489.2531 (calculated for C₂₀H₃₇N₆O₆S, 489.2495) and at m/z 493.2634 (calculated for ¹²C₁₆¹³C₄H₃₇N₆O₆S, 493.2629), respectively.

HNE Modification and HICAT Labeling of E. coli Thioredoxin

Adduction of thioredoxin (Trx) with 4-hydroxy-2-nonenal (HNE) was accomplished by dissolving 1 mg (82 nmol) of Trx in 1 mL of 10 mM sodium phosphate, pH 7.4, to which a 10-fold molar excess (13 μL) of HNE (10 mg/mL) was added. The reaction mixture was then incubated at 37° C. for 3 hours. The Trx-HNE adduct was purified by ultrafiltration (5000 molecular weight cutoff, Amicon Ultrafree-MC, Millipore, Billerica, Mass.). A 200-fold molar excess of ¹²C₄/¹³C₄-HICAT (1:1) was added to the Trx-HNE adduct reconstituted in 10 mM sodium phosphate, pH 4.7. The reaction mixture was incubated at 37° C. for 2 hours, and the hydrazone bonds were reduced at 0° C. for 1 hour with a 400-fold molar excess of sodium cyanoborohydride in ethanol followed by incubation for 2 hours at room temperature. Unreacted HICAT was removed by ultrafiltration as described above. After purification, the HICAT-labeled HNE-modified Trx adduct was digested using trypsin at a ratio of 1:50 at 37° C. for 18 hours. For the affinity enrichment of biotinylated peptides a monomeric avidin cartridge was used according to the manufacturer's instructions.

### HICAT Labeling of In Vitro HNE-Modified Mitochondrial Proteins

Rat heart mitochondria were isolated according to Suh et al. (Free Radical Biol. Med. (2003) 35:1064-1072). Mitochondria were disrupted by several freeze-thaw cycles. Soluble and insoluble protein fractions were obtained by centrifugation. Protein concentrations were determined by using Coomassie plus protein assay reagent. The soluble protein fraction in 10 mM sodium phosphate was reacted with HNE (1.5 mM) at 37° C. for 3 hours followed by purification using a Zeba desalting spin column (7000 molecular weight cutoff, Pierce Biotechnology, Rockford, Ill.). The HNE-modified mitochondrial proteins were digested with trypsin in 100 mM Tris-HCl (pH 8.0) at 37° C. for 8 hours prior to spin filtration (Amicon Ultrafree-MC centrifugal filter, 5000 molecular weight cutoff, Millipore, Billerica, Mass.). The collected peptides were treated with HICAT (5 mM) for 2 hours at 37° C. in 100 mM sodium phosphate, pH 4.7, followed by reduction of the hydrazone bonds with sodium cyanoborohydride (0.1 N) at 4° C. overnight. The excess HICAT reagents were separated from the peptides by using HPLC (Vydac C4 (250 mm×4.0 mm), flow rate 1 mL/minute, detection at 215 and 254 nm). HICAT eluted within 12 minutes with 6% CH₃CN, and peptides were eluted with a gradient from 6% to 95% CH₃CN over 20 minutes. Collected fractions were combined for subsequent lyophilization. The lyophilized sample was dissolved in loading buffer consisting of 20 mM sodium phosphate buffer with 20% CH₃CN, pH 7.2, for affinity enrichment.

Quantification labeling of in vitro HNE-modified mitochondrial proteins using ¹²C₄/¹³C₄-HICAT was carried using the same protocol as outlined above, except that aliquots of HNE-modified mitochondrial proteins were digested with trypsin prior to treatment with either ¹²C₄- or ¹³C₄-HICAT and combined after reduction with sodium cyanoborohydride.

¹²C₄- and ¹³C₄-HICAT Labeling of Cardiac Mitochondrial Proteins from Young and Old Animals

Cardiac mitochondrial samples from three young rats and three old rats were mixed and prepared as above. Aliquots of young and old rat mitochondrial proteins were digested with trypsin, then treated with ¹²C₄- and ¹³C₄-HICAT, respectively, and sodium cyanoborohydride was added. Reaction mixtures were combined, and samples were worked up as described.

### NanoHPLC

An Ultimate LC Packing system (Dionex, Sunnyvale, Calif.) coupled to a MALDI target spotter (Probot) was used. Peptide samples were loaded onto a 5 mm×0.50 mm C18 trap cartridge at a flow rate of 30 μL/minute. After 10 minutes the trap cartridge was automatically switched in-line with a 75 μm i.d.×15 cm C18 PepMap 100 column. Peptides were eluted with a gradient from 20% to 100% B over 60 minutes using as solvent A, 5% acetonitrile containing 0.1% TFA, and as solvent B, 80% acetonitrile containing 0.1% TFA. The flow rate was 0.325 μL/minute. The column effluent was mixed with R-cyano-4-hydroxycinnamic acid (2 mg/mL in 50% acetonitrile containing 0.1% TFA) via a Tee junction, and fractions (20 seconds each) were collected onto a stainless steel target plate.

### Mass Spectrometry

MALDI-MS/MS analysis was performed on an ABI 4700 Proteomics analyzer with TOF/TOF optics equipped with a Nd:YAG laser operating at a wavelength of 355 nm (Applied Biosystems, Inc., Framingham, Mass.) as previously described (Chavez et al. (2006) Anal. Chem., 78:6847-6854). Briefly, the accelerating voltage was set to 20 kV, and a collision energy of 1 kV was used. The precursor ion was selected by operating the time-gated window at approximately 3-10 Da width. Gas pressure (air) in the collision cell was set to 6×10⁻⁷ torr. Fragment ions were accelerated at 15 kV into the reflector. MALDI-MS/MS data were processed with GPS Explorer 2.0 for creating Mascot-searchable files. NanoLC-ESI-MS/MS instrumentation was essentially the same as previously described (Chavez et al. (2006) Anal. Chem., 78:6847-6854). Briefly, ESI-MS/MS was performed using a quadrupole orthogonal time-of-flight mass spectrometer (Q-TOF Ultima Global, Micromass/Waters, Manchester, U.K.) coupled to a nanoAcquity Ultra Performance LC (Waters, Milford, Mass.) and operated at 3.5 kV. A Symmetry C18 (5 μm, 180 μm i.d.×20 mm) was used as a trap column at 3 μL/min, and an in-house packed Michrom Magic C18 (75 μm i.d.×17 cm) was used for peptide fractionation. The data-dependent MS/MS mode was used with a 0.6 second survey scan and 2.4 second MS/MS scans on the three most abundant ion signals in the MS survey scan, with previously selected m/z values being excluded for 60 seconds. The collision energy for MS/MS (25-75 eV) was dynamically selected based on the charge state of the precursor ion selected. Peaklist (pkl) files were created using ProteinLynx Global Server (PLGS, Waters, Manchester, U.K.).

### Mass Spectral Data Processing and Database Searching

For protein identification, MS/MS data were searched against the mammalian SwissProt database (Taxonomy rodentia) using the Mascot search engine (Matrix Science, London, U.K.). Trypsin/P was selected as the digesting enzyme, and one missed cleavage site was allowed. The following variable modifications were considered: oxidized Met (147.04 Da, monoisotopic mass); ¹²C₄/¹³C₄-HICAT-labeled HNE-Cys, -His, and -Lys (monoisotopic masses 731.38 and 735.38, 765.43 and 769.43, 756.47 and 760.47 Da, respectively); ¹²C₄/¹³C₄-HICAT-labeled HHE-Cys, -His, and -Lys (monoisotopic masses 689.32 and 693.32, 723.37 and 727.37, 714.41 and 718.41 Da, respectively); ¹²C₄/¹³C₄-HICAT-labeled acrolein-Cys, -His, and -Lys (monoisotopic masses 631.28 and 635.28, 665.33 and 669.33, 656.37 and 660.37 Da, respectively). Searches were done with initial mass tolerance of 0.2 Da in the MS mode and 0.2 Da in the MS/MS mode. In addition, tandem mass spectra of HICAT-labeled peptides were visually inspected for preferred fragmentation patterns, such as N-terminal cleavage to Pro (P) and C-terminal cleavage to Asp (D).

### Results

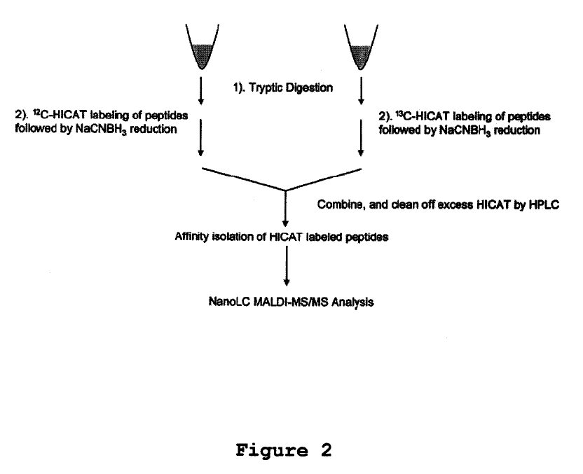

The hydrazide-functionalized isotope-coded affinity tag (HICAT) as a novel tool for enrichment and identification of low-abundance protein-oxylipid conjugates using mass spectrometry is shown in FIG. 1. The HICAT reagent used here consists of a hydrazide functional group with specificity toward aldehyde/keto groups, an isotope-coded carbon linker, and a biotin affinity tag, which can be used to characterize and quantify carbonyl (aldehyde and/or ketone)-containing proteins. The HICAT approach, applied here to the analysis of oxylipid-protein conjugates, consists of three steps (FIG. 2): (1) tryptic proteolysis of protein samples that contain oxylipid-modified proteins, followed by covalent tagging of the oxylipid-peptide conjugates by the HICAT probes, (2) samples are combined and HICAT-labeled peptides are enriched by avidin-based affinity chromatography, and (3) mass spectrometric detection of the isotopomeric HICAT-tagged peptide pairs that are common in both samples for relative quantitation and tandem mass spectrometry for peptide sequence information, the nature of the oxylipid, and the site of modification.

In proteins, products of lipid peroxidation, such as α,β-unsaturated aldehydes, are able to modify nucleophilic amino acid residues via a Michael-type addition reaction (Carini et al. (2004) Mass Spectrom. Rev., 23:281-305). To study the applicability of HICAT for selective labeling and quantification of Michael addition-type oxylipid-protein conjugates, E. coli thioredoxin was used as a model system. Trx contains three nucleophilic residues, His-6, Cys-32, and Cys-34, but in the oxidized form Cys-32 and Cys-34 are disulfide-linked and not available for modification. Thus, His-6 is the only residue available for conjugation reactions with 2-alkenals.

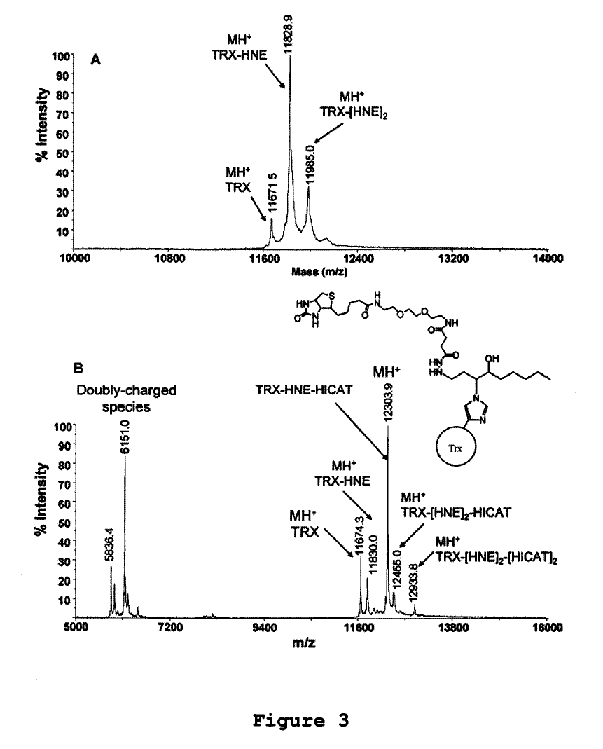

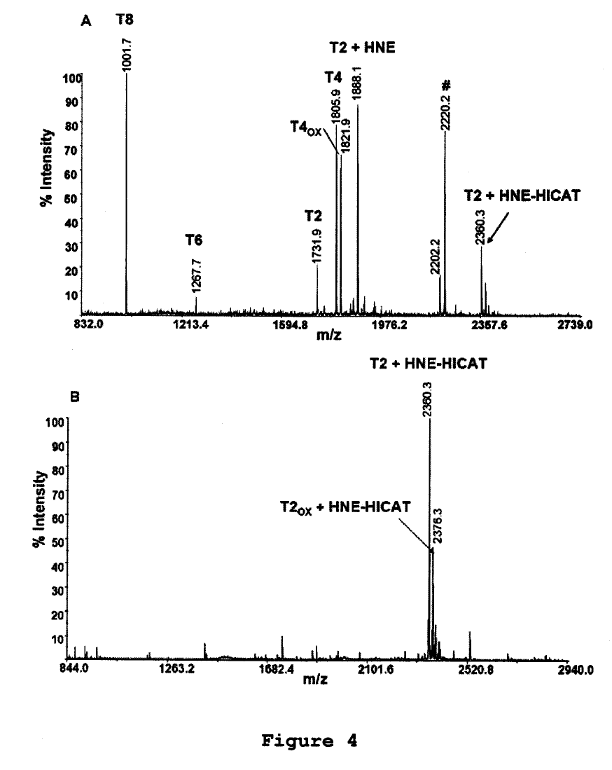

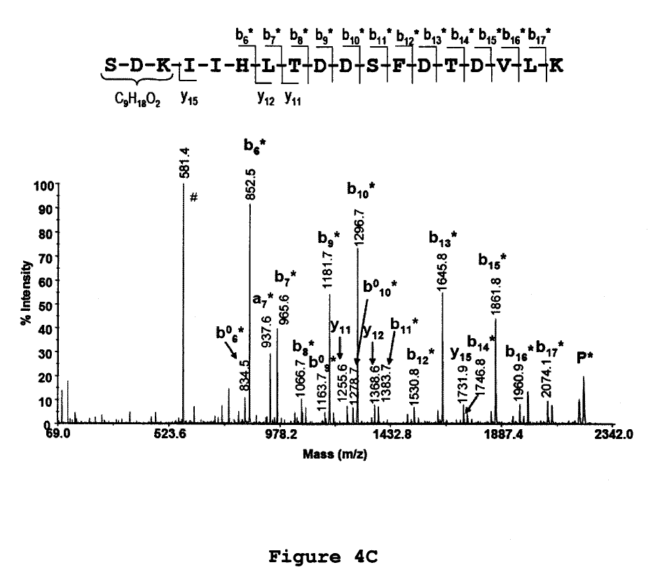

Matrix-assisted laser desorption/ionization mass spectrometry (MALDI-MS) of the reaction product of HNE with Trx in its oxidized state confirmed that mostly a monoadduct product was obtained (FIG. 3A). The mass spectral peak at m/z 11 671.5 represents the unmodified Trx, and the ion peak at m/z 11 828.9 corresponds to the addition of one HNE molecule (Δm 157.4). A small fraction of the protein was found to be modified by two molecules of HNE (TRX-(HNE)₂, MH⁺ 11 985 Da). Subsequent reaction with ¹²C-HICAT was followed by NaCNBH₃ reduction to convert the hydrazone bond into the more stable substituted hydrazide-type linkage. The reaction was monitored by MALDIMS, and HICAT labeling was confirmed by the mass spectral peak at m/z 12 303.9 representing Trx-HNE-HICAT (FIG. 3B). The minor peaks at m/z 12 457 and 12 934 indicate labeling of TRX-(HNE)₂ by one and two HICAT molecules, respectively (FIG. 3B). The peak areas in the MALDI mass spectra were used to obtain estimates of the labeling efficiency of the HICAT probe. Labeling efficiencies reached approximately 65-75%. However, caution should be used when using of ion intensities observed in MALDI-MS experiments for deducing quantitative information. Thus, the percentage of recovery for the HICAT conjugate is an approximated estimate. A critical step in the HICAT labeling approach is the reduction of the hydrazone bond with NaCNBH₃. At the applied conditions, this reduction step proceeds with about 50% efficiency as deduced from the preliminary chemical reaction studies in which the HICAT coupling and subsequent reduction of HNE-modified model peptides was analyzed. A similar conclusion can also be drawn from the MALDI-MS data of the tryptic digest of the HICAT-labeled HNE-Trx conjugate presented in FIG. 4A, keeping in mind that biotin-tagged peptides commonly show reduced ion abundances in MALDI-MS experiment compared to non-biotin-containing peptides.

The product was digested with trypsin, and the HICAT-labeled HNE-peptide conjugates were isolated by affinity chromatography (FIG. 4). The enriched peptide with m/z 2360.4 was subjected to tandem mass spectrometry to confirm HICAT labeling of the HNE-modified His residue at position 6. Collision-induced fragmentation of the peptide at m/z 2360.4 yielded a series of b-type ions and an intense His-HNE-HICAT immonium ion at m/z 738.3 (FIG. 5). The fragment ions F1, F2, and F3 are indicative of some degree of side-chain fragmentation of the HICAT moiety. Ions at m/z 1904.0 and m/z 1986.0 result from neutral loss of the F3 and F2 moiety from the precursor ion, MH⁺ 2360.4, respectively. Neutral loss of the HICAT-HNE moiety yields the ion at m/z 1731.9.

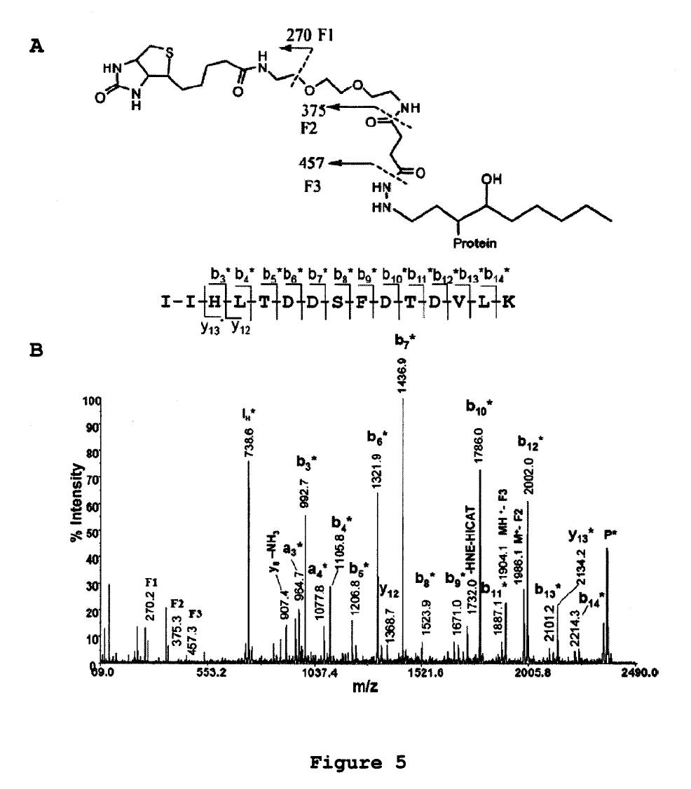

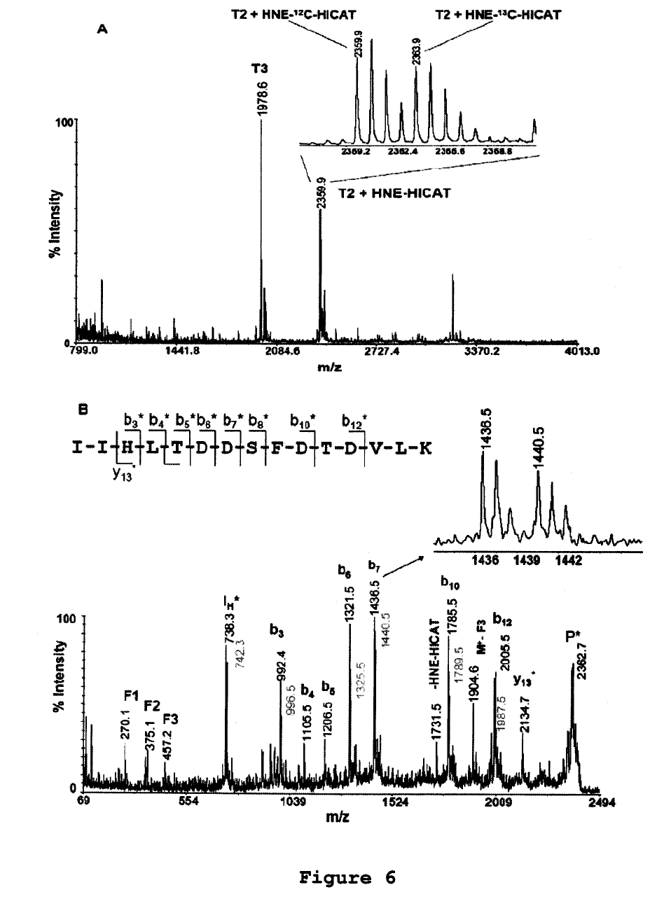

Mass spectrometric analysis of the tryptic digest of TRX-HNE conjugate labeled with equal amounts of ¹²C₄-HICAT and ¹³C₄-HICAT showed in the MS survey scan the His-containing peptide as a paired set of mass peaks with a mass difference of 4 Da, consistent with the mass difference between the two HICAT isotopomers, and the expected 1:1 ratio for the peptide ions at m/z 2360 and m/z 2364 (FIG. 6A). When the ¹³C₄-HICAT-labeled peptide ion with m/z 2364 was selected for collision-induced dissociation, it yielded an MS/MS spectrum (FIG. 6B) comparable to that of the ¹²C₄-HICAT-labeled peptide, revealing the site of modification at His-6 when His-HNE-¹³C₄-HICAT and His-HNE-¹²C₄-HICAT were entered as modified His residues in the Mascot search. Examination of the bₙ fragment ions shows both the ¹²C- and ¹³C-labeled fragment ions; this is due to the fact that the time-gated window used for of the precursor ion selection was too wide to exclude the ¹²C-labeled peptide with m/z 2360.

To demonstrate the utility of the HICAT reagent for the identification of oxylipid-modified mitochondrial proteins in complex samples, soluble protein fractions obtained from rat heart mitochondria were treated with HNE. Initially, after removal of excess HNE, the sample was treated with HICAT in phosphate buffer pH 4.7 for 2 hours at 37° C., and, then, treated with NaCNBH₃ to reduce the resultant hydrazone bond. Unfortunately, precipitation of mitochondrial proteins was observed at this pH. Other conditions such as pH 7.4 with large excess HICAT or extended reaction time were also tested, without success. Thereafter, another approach to overcome the precipitation issue was developed (FIG. 2). After in vitro modification with HNE, the mitochondrial proteins were first digested with trypsin and subsequently labeled with HICAT. Excess HICAT and NaCNBH₃ were removed by HPLC, and the isolated peptide fractions were subjected to affinity chromatography. Enriched fractions were further processed by reversed-phase nanoLC and analyzed by MALDI-MS/MS in conjunction with Mascot database searching. A total of 30 HICAT-labeled, HNE-conjugated peptides from 18 mitochondrial proteins were identified based on the Mascot score and the assignment of bₙ and yₙ ions (Table 1). All identified peptides were found in the affinity-enriched fraction. Additional HICAT-labeled, HNE-conjugated peptides from mitochondrial proteins with Mascot score are provided in Table 2.

TABLE 1

Selected HICAT-labeled HNE in vitro-modified
peptides identified by Mascot search of MS/MS Data.

| | | | |
|---|---|---|---|
| | | Mascot | SEQ ID |
| Protein Name | Labeled Peptides | Score | NO: |

| | | | |
|---|---|---|---|
| ATP synthase β chain | LVLEVAQH*LGESTVR^{b,c,d} | 48 | 1 |
| (ATPB_RAT) | IMDPNIVGSEH*YDVAR^{b} | 39 | 2 |
| | AH*GGYSVFAGVGER^{b,c,d} | 68 | 3 |
| | EGNDLYH*EMIESGVINLK^{b} | 35 | 4 |
| ATP synthase α chain | H*ALIIYDDLSK^{b,c,d} | 58 | 5 |
| (ATPA_RAT) | | | |
| ATP synthase B chain | H*VIQSISAQQEK^{b,c} | 40 | 6 |
| (AT5F1_RAT) | H*YLFDVQR^{b,c} | 40 | 7 |
| | LDYH*ISVQDMMR^{b,c} | 32 | 8 |
| ATP synthase D chain | NC*AQFVTGSQAR^{b,c,d} | 54 | 9 |
| (ATP5H_RAT) | SWNETFH*TR^{b} | 29 | 10 |
| ATP synthase γ chain | TH*SDQFLVSFK^{b,c} | 42 | 11 |
| (ATPG_RAT) | HLIIGVSSDR^{b,c} | 26 | 12 |
| ATP synthase oligomycin/ | GEVPC*TVTTAFPLDEAVLSELK^{b,c} | 49 | 13 |
| sensitivity conferral protein | | | |
| (ATPO_RAT) | | | |
| ADP, ATP carrier protein 1 | LLLQVQH*ASK^{b,c} | 26 | 14 |
| (ADT1_RAT) | GADIMYTGTVDC*WR^{c} | 19 | 15 |
| acyl-CoA dehydrogenase, long | IFSSEH*DIFR^{b,c} | 23 | 16 |
| chain specific (ACADL_RAT) | C*IGAIAMTEPGAGSDLQGVR^{c} | 48 | 17 |
| 3-ketoacyl-CoA thiolase | LEDTLWAGLTDQH*VK^{b} | 35 | 18 |
| (THIM_RAT) | | | |
| aconitate hydratase (ACON_RAT) | IVYGH*LDDPANQEIER^{b} | 21 | 19 |
| cytochrome c oxidase subunit IV | DYPLPDVAH*VK^{b,c} | 40 | 20 |
| isoform 1 (COX41_RAT) | | | |
| cytochrome c oxidase polypeptide | LVPYQMVH*^{b,c} | 20 | 21 |
| Vb (COX5B_RAT) | | | |
| cytochrome c oxidase polypeptide | LFQEDNGMPVH*LK^{b} | 20 | 22 |
| VIIa-liver/heart (CX7A2_RAT) | | | |
| cytochrome c oxidase polypeptide | SH*YEEGPGK^{b,c} | 21 | 23 |
| VIIc (COX7C_RAT) | | | |
| ubiquinol-cytochrome-c reductase | NALANPLYC*PDYR^{b,c} | 32 | 24 |
| complex core protein 2 | | | |
| (UQCR2_RAT) | | | |
| creatine kinase (KCRS_RAT) | H*NGYDPR^{b,c} | 20 | 25 |
| malate dehydrogenase | GC*DVVVIPAGVPR^{b,d} | 62 | 26 |
| (MDHM_RAT) | ETEC*TYFSTPLLLGK^{b,c,d} | 48 | 27 |
| | VNVPVIGGH*AGK^{b} | 30 | 28 |
| | H*GVYNPNK^{b,c,d} | 26 | 29 |
| succinate dehydrogenase | VSDAISTQYPVVDH*EFDAVVVG | 38 | 30 |
| flavoprotein subunit | AGGAG LR^{b,c} | | |
| (DHSA_RAT) | | | |

An * marks the HICAT-labeled HNE in vitro-modified residue.
All MS/MS spectra were visually inspected and fragment ions annotated manually in addition to the automated annotations generated by Mascot.
bPeptide identified from ¹²C₄-HICAT tagging experiments.
cPeptide identified from ¹²C₄/¹³C₄HICAT tagging experiments.
dPeptide for which both MALDI- as well as ESI-MS/MS data are available.
eThis peptide is derived from the C-terminus of the cytochrome c oxidase polypeptide Vb chain.

TABLE 2

HICAT-labeled HNE in vitro-modified peptides
identified by MASCOT search of MS/MS data.

| | | | SEQ |
|---|---|---|---|
| | | Mascot | ID |
| Protein Name | Labeled Peptides | Score | NO: |
| Aconitase | VGLIGSCTNSSYEDMGR | 35 | 36 |
| | EHAALEPR | 38 | 37 |
| Propionyl-CoA | HAALLGGGQR | 34 | 38 |
| carboxylase β-chain | | | |
| HADH-ubiquinone | NYPEGHR | 34 | 39 |
| oxidoreductase, | | | |
| 24 kDa subunit | | | |
| 3-ketoacyl-CoA | KHNFTPLAR | 25 | 40 |
| thiolase | | | |
| Succinyl-CoA ligase | MGHAGAIIAGGK | 25 | 41 |
| Trifunctional | THINYGVK | 22 | 42 |
| enzyme alpha | | | |
| succinate dehydrogenase | GVIALC*IEDGSIHR | 17 | 43 |
| flavoprotein | | | |
| subunit (DHSA_RAT) | | | |

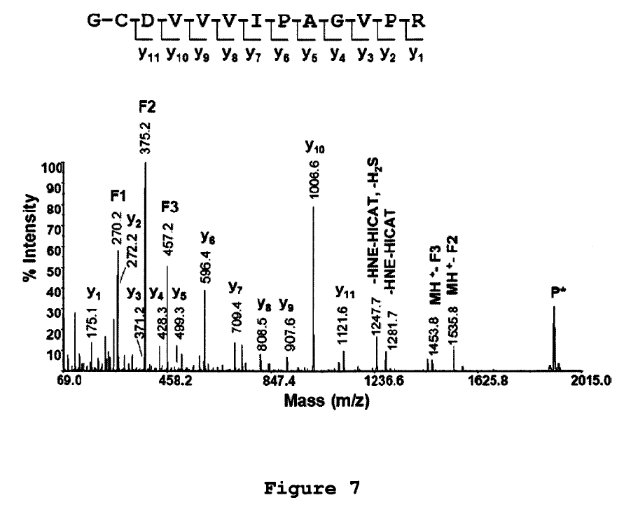

For example, the high-energy collision-induced fragmentation of the peptide GCDVVVIPAGVPR (m/z 1909.1; SEQ ID NO: 26), from malate dehydrogenase (MDHM_RAT), resulted in an almost complete y ion series (y₁-y₁₁). This spectrum yielded the highest ion score of the HICAT-HNE-modified peptides, namely, 62 (Table 1 and FIG. 7). The presence of an Asp residue in this peptide causes the relative predominance of the y₁₀ fragment ion. The enhanced cleavage probability of the peptide amide bond C-terminal to the Asp residue in this peptide is particularly pronounced due to the presence of the C-terminal Arg residue sequestering the proton (Herrmann et al. (2005) J. Am. Soc. Mass Spectrom., 16:1067-1080; Gu et al. (2000) Anal. Chem., 72:5804-5813). The intense y₆ ion is indicative of the preferred fragmentation of the amide bond N-terminal to the Pro residue (Wysocki et al. (2000) J. Mass Spectrom., 35:1399-1406). Besides the sequence-specific peptide fragment ions, neutral loss ions at 1454.8 and 1535.8 were observed, as well as HICAT-specific fragment ions annotated F1 (m/z 270 Da), F2 (m/z 375 Da), and F3 (m/z 457 Da).

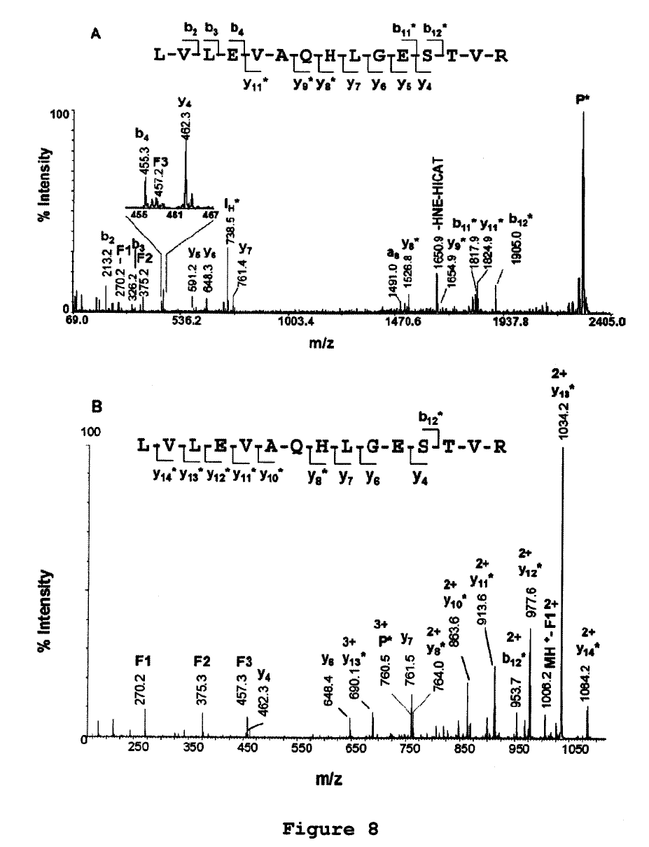

FIG. 8A shows the MALDI tandem mass spectrum of an HICAT-labeled HNE-modified peptide ion with MH⁺ 2279.3 from ATP synthase β-chain (ATPB_RAT), which yielded a Mascot score of 48. This peptide contains 15 amino acids, but only one nucleophilic site, namely, the His residue which was found to be modified by the HNE-HICAT moiety. Interesting features of this spectrum include a few b ions (b₂-b₄, b₁₁, and b₁₂) and an extended y ion series (y₁-y₈ and y₁₁) The m/z difference of 765.4 between the y₈ and the y₉ fragment ion confirmed the presence of a His-HNE-HICAT residue in position 8. The fragment ion with m/z 1680.9 indicates loss of the HICAT-HNE moiety from the precursor ion MH⁺ m/z 2279.3. The HICAT-HNE-modified His immonium ion is visible at m/z 738.5.

This peptide from ATP synthase β-chain, LVLEVAQH*LGESTVR (SEQ ID NO: 1), was also identified by ESI-MS/MS using a quadrupole time-of-flight-type instrument (FIG. 8B). The triply charged molecular ion [M+3H]³⁺ (m/z 760.4) of the HICAT-HNE-modified peptide was selected as the precursor ion. An almost complete yₙ fragment ion series was observed. The m/z difference between the y₇ ion (at m/z 761.4) and the y₈ ion (at m/z 1526.8) locates the HICAT-HNE moiety on the His residue. This spectrum yielded a Mascot score of 45. Similar to the observations made in MALDI-MS/MS spectra, ions that indicate neutral loss or side-chain fragmentation of the HICAT-HNE moiety were also observed. The HICAT-specific fragment ions, F1, F2, and F3, were consistently observed in MS/MS spectra and, thus, could be useful potentially as “indicator” ions aiding in the identification of HICAT-labeled peptides or in precursor ion scanning experiments. However, the caveat is that nonpeptide ions without annotation affect the Mascot score adversely. Nonpeptide fragment ions were also observed for peptides modified by the commercially available ICAT probe (Borisov et al. (2002) Anal. Chem., 74:2284-2292).

To demonstrate the feasibility of using stable-isotope-coded HICAT reagents to selectively target and quantify mitochondrial oxylipid-protein conjugates, mitochondrial protein extracts were treated with HNE in vitro. After tryptic digestion, aliquots of the digest were reacted separately with excess ¹²C₄-HICAT and ¹³C₄-HICAT followed by NaCNBH₃. The two aliquots were combined prior to HPLC purification (FIG. 2). The peptide-containing fractions were combined, lyophilized, and subjected to affinity chromatography. The enriched fraction was further processed by reversed-phase nanoLC and analyzed by MALDI-MS/MS in conjunction with Mascot database searching (FIG. 6). Because the time gate of the TOF/TOF ion optics is operated at a window of approximately 4-10 Da for precursor ion selection, the tandem mass spectra exhibited fragment ions from both ¹²C₄-HICAT- and ¹³C₄-HICAT-labeled precursor ions, which generally resulted in lower Mascot scores.

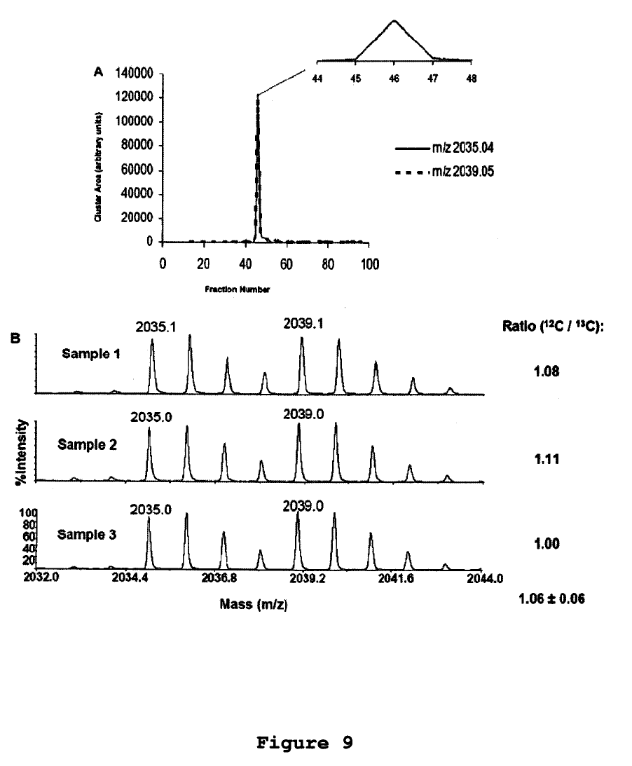

For determining the isotopomeric ratios of ¹²C₄/¹³C₄-HICAT-tagged peptide pairs, ion traces observed in survey scans were evaluated to calculate the ratios according to the isotopic cluster area of each ¹²C₄/¹³C₄-HICAT-tagged peptide pair. Three samples were prepared and used to demonstrate the precision and accuracy of this method. As an example, in FIG. 9, the isotope cluster observed for the peptide AH*GGYSVFAGVGER (SEQ ID NO: 3) from ATP synthase β-chain (ATPB_RAT) is depicted. The peak area ratio of the ¹²C/¹³C pair was determined with a variability of 5.7% and an average ratio of 1.06±0.06 (mean±SD) from three independently prepared samples. Peak area ratios for a selection of 16 ¹²C₄/¹³C₄-HICAT-HNE-modified peptides are listed in Table 3. The average ratios of these ¹²C/¹³C-tagged peptides range from 1.01±0.08 for the peptide from ATP synthase R-chain (ATPA_RAT) to 1.15±0.30 for the peptide derived from the longchain-specific acyl-CoA dehydrogenase (ACADL_RAT). The overall average ratio for these ¹²C/¹³C-tagged peptides was 1.08±0.08. The overall variability in peptide quantification for these peptides was 7.4%. The mean values and variability for the peak area ratios for isotopomeric peptide ions are comparable to results reported for commercially available cleavable ICAT reagents (Hansen et al. (2003) Mol. Cell. Proteomics, 2:299-314).

TABLE 3

Peptide peak area ratios observed in MALDI-MS
survey scans of 12C4- and 13C4-HICAT-labeled HNE-
peptide conjugates found in mitochondrial protein
extracts treated with HNE.

Observed Ratio (¹²C/¹³C)

Samples

| | | | | | | |
|---|---|---|---|---|---|---|
| Protein | | | | | Average ± | SEQ ID |
| Name | Labeled Peptides | 1 | 2 | 3 | SD | NO: |

| | | | | | | |
|---|---|---|---|---|---|---|
| ATPB_RAT | AH*GGYSVFAGVGER | 1.08 | 1.11 | 1.00 | 1.06±0.06 | 3 |
| | LVLEVAQH*LGESTVR | 1.15 | 1.14 | 0.90 | 1.06±0.14 | 1 |
| ATPA_RAT | H*ALIIYDDLSK | 1.10 | 0.98 | 0.95 | 1.01±0.08 | 5 |
| AT5F1_RAT | H*YLFDVQR | 1.16 | 0.99 | 0.97 | 1.04±0.10 | 7 |
| | H*VIQSISAQQEK | 1.23 | 1.06 | 1.07 | 1.12±0.10 | 6 |
| | LDYH*ISVQDMMR | 1.13 | 1.05 | 1.09 | 1.09±0.04 | 8 |
| ATP5H_RAT | NC*AQFVTGSQAR | 1.23 | 1.15 | 1.01 | 1.13±0.11 | 9 |
| ATPG_RAT | TH*SDQFLVSFK | 1.16 | 1.23 | 0.95 | 1.11±0.15 | 11 |
| ATPO_RAT | GEVPC*TVTTAFPLDE | 1.00 | 1.25 | 0.96 | 1.07±0.16 | 13 |
| | AVLSELK | | | | | |
| ADT1_RAT | GADIMYTGTVDC*WR | 1.16 | 1.12 | 0.96 | 1.08±0.11 | 15 |
| | LLLQVQH*ASK | 1.03 | 1.00 | 1.08 | 1.04±0.04 | 14 |
| ACADL_RAT | C*IGAIAMTEPGAGS | 1.33 | 1.32 | 0.81 | 1.15±0.30 | 17 |
| | DLQGVR | | | | | |
| KCRS_RAT | H*NGYDPR | 1.07 | 1.20 | 0.86 | 1.04±0.17 | 25 |
| MDHM_RAT | ETEC*TYFSTPLLLGK | 1.30 | 1.05 | 1.10 | 1.15±0.13 | 27 |
| | H*GVYNPNK | 1.08 | 1.10 | 1.16 | 1.11±0.04 | 29 |
| UQCR2_RAT | NALANPLYC*PDYR | 1.13 | 1.08 | 1.01 | 1.07±0.06 | 24 |
| | | 1.15 ± | 1.11± | 1.00± | | |
| | | 0.09 | 0.09 | 0.09 | | |

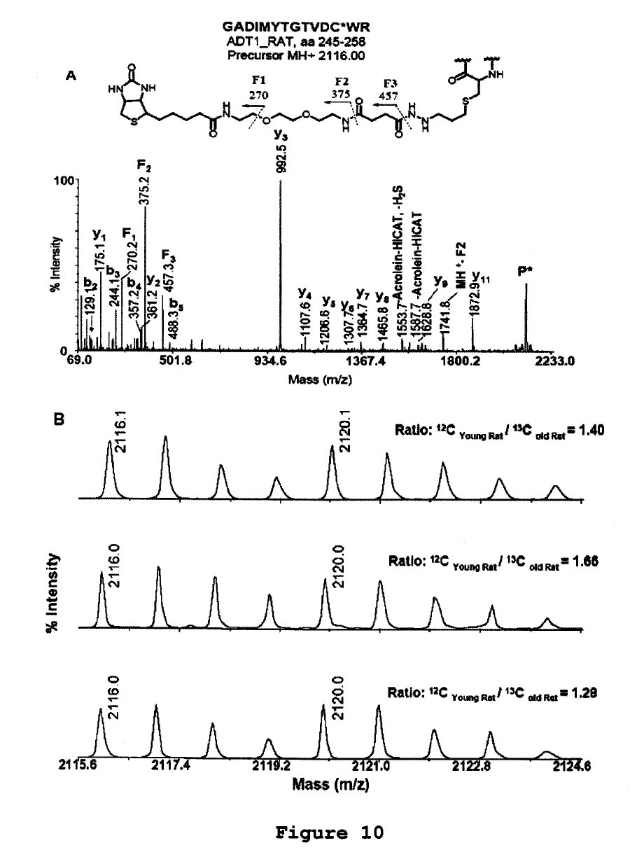

In initial experiments using the HICAT strategy, several in vivo oxylipid-protein conjugates were found in cardiac mitochondria proteins. As a prominent example, the identification of the ADP/ATP translocase 1 (ADT1_RAT) as an in vivo target of lipid peroxidation products is discussed. The tandem mass spectrum of the ¹²C₄-HICAT-labeled acrolein-conjugated peptide (MH⁺ 2116.00) is depicted in FIG. 10. The observed yₙ fragment ions (y₁-y₉, y₁₁) identify the peptide as the partial sequence 245-258 of ADT1_RAT. The presence of an Asp residue in this peptide causes the relative predominance of the y₃ fragment ion. The fragment ions at m/z 1587.7 and 1553.7 are in agreement with previous observations and are related to the losses of the HICAT-acrolein moiety. Because the identified peptide contains only a single nucleophilic residue, namely, Cys-256, the tandem mass spectral data support the notion that Cys-256 is an in vivo target site for acrolein conjugation. Noteworthy, the same peptide was also found to be modified by 4-hydroxy-2-hexenal in vivo as well as by HNE in the in vitro experiments (Table 1). This particular peptide modified by acrolein was found in mitochondrial samples obtained from both young and old rat heart with a ¹²C₄/¹³C₄-HICAT ratio of 1.45±0.19 (FIG. 10). The in vivo data presented here demonstrate the potential of the HICAT strategy for the analysis of oxylipid-proteins in biological samples.

In this study, a new hydrazide-functionalized isotope-coded affinity tag (HICAT) for the covalent labeling and quantification of oxylipid-modified proteins and peptides in complex samples is provided. The methodology allows the affinity enrichment of biotinylated oxylipid peptide conjugates and enables their identification and characterization by tandem mass spectrometry. Although this study focused on proteins modified by 2-alkenals, the HICAT probes are generally applicable to the large and diverse group of carbonylated proteins (Mirzaei et al. (2006) Anal. Chem., 78:770-778; Mirzaei et al. (2005) Anal. Chem., 77:2386-2392). HICAT's potentials as an analytical tool for the identification and relative quantification of oxylipid-protein conjugates in complex samples was demonstrated for mitochondrial in vitro protein targets of HNE. Furthermore, the successful application of the HICAT strategy for the identification and quantitation of in vivo oxylipid-protein conjugates in cardiac mitochondrial protein preparations indicate that HICAT is a useful tool in oxidative stress-related research in which the detailed characterization and quantitation of oxylipid-modified proteins and other carbonyl (aldehyde and/or ketone)-containing proteins is particularly relevant.

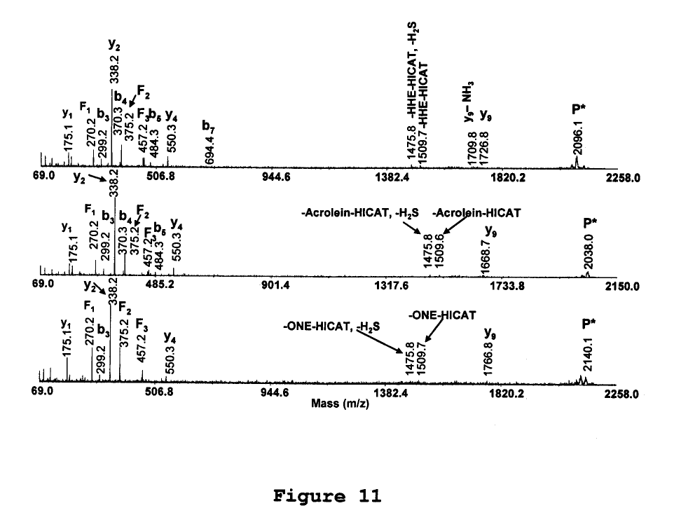

As shown hereinabove, HICAT labeling in combination with tandem mass spectrometry identified several endogenous oxylipid-modified mitochondrial proteins. Notably, distinct nucleophilic sites were found to be modified by several reactive lipid peroxidation products. For example, Cyst 191 in the peptide that originated from the Complex III core 2 polypeptide chain (UQCR2) was found to be modified by acrolein, 4-hydroxy hexenal (HHE), and 4-oxononeal (ONE) (see FIG. 11). Another interesting case demonstrating the chemical diversity of oxidation to proteins by reactive aldehydes is the mitochondrial inner membrane protein ADP/ATP translocase (ADT1_RAT). In this protein, Cys-256 was found as adduct with acrolein, HHE, and ONE. Other mitochondrial proteins that were identified as targets of acrolein adduction using HICAT were malate dehydrogenase and the flavoprotein subunit of the succinate dehydrogenase (Complex II).

Thus, oxylipid-modified peptides that originated from numerous unique polypeptides and protein were identified and characterized using aldehyde-specific chemical probes, ARP and HICAT, and hydrazide-functionlized beads. Of these oxylipid conjugates roughly 40% were caused by acrolein, approximately the same occurrence was observed for HHE and the remaining modifications were caused by ONE and HNE. Approximately two third of the found modification sites were cysteine residues and the remaining one third where histidine residues. Several of these endogenous sites were found modified by multiple reactive lipid peroxidation products and these sites were considered as “hot spots” of modification by aldehydic lipids, e.g. in Complex III, Cys-191 in the partial sequence a 170 to 182 of the core protein 2 (UQCR2_RAT), in Complex IV, His-20 of the subunit VIa (CX6A2_RAT), Cys-166 of the long-chain specific acyl-CoA dehydrogenase (ACADL_RAT) and Cys-256 of the ADP/ATP translocase (ADT1_RAT).

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A compound for the detection of at least one carbonyl group in a polypeptide, wherein said compound comprises:
a) a carbonyl reactive moiety, wherein said carbonyl reactive moiety forms a bond with said polypeptide via the carbonyl group;
b) an affinity tag; and
c) a linker region which operably links the carbonyl reactive moiety to the affinity tag;
wherein said carbonyl group is an aldehyde or a ketone.

2. The compound of claim 1, wherein said carbonyl reactive moiety is a hydrazide or a hydroxylamine.

3. The compound of claim 1, wherein said affinity tag is biotin.

4. The compound of claim 1, wherein said linker region comprises at least one stable isotope.

5. The compound of claim 4, wherein said stable isotope is ¹³C.

6. A composition comprising at least one compound of claim 1 and a carrier.

7. A method for detecting at least one carbonyl group on a polypeptide, said method comprising:
a) obtaining a composition comprising polypeptides,
b) fragmenting the polypeptides in the sample into peptides,
c) contacting the peptides with at least one compound of claim 1, and
d) isolating peptides linked to the compound by affinity chromatography,
wherein the presence of peptides linked to the compound indicates the presence at least one carbonyl group on the polypeptide which comprises the peptide; and wherein said carbonyl group is an aldehyde or a ketone.

8. The method of claim 7, further comprising determining the sequence of the peptide linked to the compound.

9. The method of claim 8, further comprising searching the obtained sequence against a database of sequences to identify the polypeptide which comprises the modified peptide.

10. The method of claim 7, wherein step c) is performed by contacting one portion of the composition comprising said peptides with a compound which does not comprise a stable isotope and contacting the other portion of the composition comprising said peptides with a compound which comprises a stable isotope.

11. The method of claim 11, further comprising quantitating the amount of polypeptides comprising a carbonyl group by performing isotope dilution mass spectrometry.

12. The method of claim 7, wherein the fragmenting in step b) is performed by tryptic digestion.

13. The method of claim 7, wherein step c) is performed before step b).

14. A kit comprising at least one of the compositions of claim 6.
